(19) United States
(12) Patent Application Publication
(10) Pub. No.: US 2018/0224411 A1
(43) Pub. Date: Aug. 9, 2018

(54) BOTANICAL IDENTIFICATION METHOD AND SYSTEM

(71) Applicant: SCIENTIFIC HOLDINGS, LLG, Monrovia, CA (US)

(72) Inventors: Jeffrey Charles Raber, Pasadena, CA (US); Sytze Elzinga, Denver, CO (US); Raquel Keledjian, Glendale, CA (US)

(21) Appl. No.: 15/749,358

(22) PCT Filed: Jul. 30, 2016
(86) PCT No.: PCT/US16/44929

§ 371 Date: Jan. 31, 2018

Related U.S. Application Data
(60) Provisional application No. 62/282,431, filed on Jul. 31, 2015.

(51) Int. Cl. G01N 30/88; G01N 30/68; A61K 31/352

(52) U.S. Cl. CPC G01N 30/88; G01N 2030/884; A61K 31/352; G01N 30/68

(57) ABSTRACT

The disclosure provides a method for extracting, separating, and identifying compounds from natural products, such as cannabis or hops, where separation uses gas chromatography (GC). The method includes spiking the composition to be identified with at least two markers that bracket the migration position of at least one of the compounds to be identified, where GC method uses more than one ramping step.

### FIELD OF THE DISCLOSURE

The disclosure relates to methods for separating, purifying, and identifying terpenes, cannabinoids, and related compounds, and using chromatographic and computational methods for categorizing botanical varietals.

### BACKGROUND OF THE DISCLOSURE

Profiling medicinal cannabis by analyzing only THC and CSD is exceptionally limited for effective determination of the optimal cannabis strain for a given patient. The mixture of relevant compounds in cannabis is far more complex than only the one or two cannabinoids that provide therapeutic effect. The major cannabinoids from Cannabis saliva are: cannabidiol (CBD), cannabichromene (CBC), cannabigerol (CBG), delta9-tetrahydrocannabinol (THC), and cannabinol (CBN) (Appendino et al (2008) J. Nat. Prod. 71:1427-1430). Hence, there has been some interest in procedures for identifying several cannabinoids in cannabis, as well as the terpenes that contribute to the physiological effects of cannabis.

Terpenes modify and modulate the effects of THC and other cannabinoids and impact the overall medicinal properties of the particular cultivar. Terpenes are also predominant players in the smell and taste of medicinal cannabis. Moreover, terpenes alone, when inhaled from the ambient air, can influence animal and human behavior. Physiological effects can be detected when inhaled from ambient air, where the result is serum levels in the single digit ng/mL range (see, US 2016/0080285 of Elzinga and Raber).

Terpenes display unique therapeutic effects that may contribute to the overall effects of medicinal cannabis. The synergy of terpenes and cannabinoids are likely responsible for providing the effective treatment of pain, anxiety, epilepsy, inflammation, depression, and infections (McPartland and Russo (2001) J. Cannabis Ther. 1:103-132).

The term “entourage effect” refers to the influence of the combination of cannabinoids and terpenes that results in synergic effects on physiology. It is recognized that, “[t]his type of synergism may play a role in the widely held . . . view that in some cases plants are better drugs than the natural products isolated from them. Support derives from studies in which cannabis extracts demonstrated effects two to four times greater than THC” (Russo (2011) Brit. J. Pharmacol. 163:1344-1364). Moreover, it is recognized that cannabis produces its medical effects, “by virtue of the concentration and balance of various active ingredients, especially the cannabinoids . . . but also . . . a wide range of terpenoids and flavonoids” (Corral (2001) J. Cannabis Therapeutics. vol. 1, issue 3.4).

For medicinal cannabis patients to receive the proper medication, it is of critical importance to find the right strain of cannabis, and to find the right product, in order to meet the medical needs of the patient. The health care provider or the patient will need to understand the products terpene content, in order to seek and take advantage of the complete entourage effect being delivered by a particular selected cannabis. Clinical trials have established that cannabis, or formulations derived from cannabis, can improve neuropathic pain of multiple sclerosis, improve appetite and sleep quality in cancer patients, relieve pain in fibromyalgia patients, and serve as an anti-emetic for chemotherapy induced nausea and vomiting (see, Health Canada (February 2013) Information for Health Care Professionals. Cannabis (Marihuana, Marijuana) and the Cannabinolds (152 pages)).

It is recognized that, “lain important question that remains to be answered is which of the many varieties of cannabis should be made available for medical use. Drug varieties of cannabis are commonly distinguished through the use of popular names . . . it is unclear whether such classification reflects any relevant differences in chemical composition” (Hazekamp at al (2012) Drug Testing Analysis. 4:660-667). The present disclosure fulfils this unmet need by providing an efficient method and system for separating and identifying cannabinoids and terpenes. Moreover, the present disclosure fulfils the need to identify a complex mixture of terpenes and cannabinoids by providing a method for extracting, separating, and identifying compounds from natural products, using gas chromatography (GC), where the method includes spiking the extract or complex mixture with at least two markers that bracket the migration position of at least one of the compounds to be identified, and where the GC method uses more than one ramping step.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference.

The terms “adapted to,” “configured for,” and “capable of,” mean the same thing. Where more than one of these terms are used in a claim set, it is the case that each and every one of these terms, as they might occur, means, “capable of.”

The term “GC run” refers, by way of a non-limiting example, to the process where a sample comprising at least one cannabinoid, terpene, or a combination of terpenes and cannabinoids, is introduced into a gas chromatography (GC) apparatus, subjected to ramping procedures, where migration occurs and migration data is collected, and where the GC apparatus is returned to a condition suitable for analysis of a second sample of cannabinoids or terpenes.

The founder of terpene chemistry is Otto Wallach who received the Nobel Prize in 1910 (Christmann (2010) Angew Chem. Int. Ed. Engl. 49:9580-9586). The terpenes are biosynthesized from units of isoprene, which can be linked to form linear chains or rings. In increasing length, the terpenes include hemiterpenes (single isoprenoid unit), monoterpenes (two units), sesquiterpenes (three units), diterpenes (four units), sesterterpenes (five units), triterpenes (six units), and so on. Non-aromatic terpenes include vitamin A, vitamin K, and the taxanes. The taxanes (diterpenes), such as paclitaxel, are renowned for their use in treating cancer (Heinig and Jennewein (2009) African J. Biotech. 8:1370-1385). Terpenes in cannabis have been described. See, Flores-Sanchez and Verpoorte (2008) Phytochem. Rev. 7:615-639, and US2015/0080265 of Elzinga and Raber and US2015/0152018 of Raber and Elzinga, each of which is incorporated herein in its entirety.

Some examples of terpenes, and their classification, are as follows: Hemiterpenes: Examples of hemiterpenes, which do not necessarily have an odor, are 2-methyl-1,3-butadiene, hemialboside, and hymenoside; Monoterpenes: pinene; alpha-pinene, beta-pinene, cis-pinane, trans-pinane, cis-pinanol, trans-pinanol (Erman and Kane (2008) Chem. Biodivers. 6:910-919), limonene: linalool; myrcene; eucalyptol; alpha-phellandrene; beta-phellandrene; alpha-ocimene; beta-ocimene, cis-ocimene, ocimene, delta-3-carene; fenchol; sabinene, borneol, isoborneol, camphene, camphor, phellandrene, alpha-phellandrene, alpha-terpinene, geraniol, linalool, nerol, menthol, myrcene, terpinolene, alpha-terpinolene, beta-terpinolene, gamma-terpinolene, delta-terpinolene, alpha-terpineol, trans-2-pinanol, Sesquiterpenes: caryophyllene; beta-caryophyllene, caryophyllene oxide, humulene, alpha-humulene, alpha-bisabolene; beta-bisabolene; santalol; selinene; nerolidol, bisabolol; alpha-cedrene, beta-cedrene, beta-eudesmol, eudesm-7(11)-en-4-ol, selina-3,7(11)-diene, guaiol, valencene, alpha-guaiene, beta-guaiene, delta-guaiene, guaiene, farnesene, alpha-farnesene, beta-farnesene, elemene, alpha-elemene, beta-elemene, gamma-elemene, delta-elemene, germacrene, germacrene A, germacrene B, germacrene C, germacrene D, germacrene E. Diterpenes: oridonin, Triterpenes: ursolic add; oleanolic acid; [0012] “1.5 ene”: guaia-1(10),11-diene can be characterized as a 1.5 ene. Guaia-1(10),11-diene is halfway between a monoterpene and diterpene, in terms of how many isoprenoid units are present. Monoterpene is C₁₀H₁₆, and diterpene is C₂₀H₃₂. Guaia-1(10),11-diene is C₁₅H₂₄. Isoprene is C₆H₈ (two double bonds).

Cannaboids and related compounds can be identified by the methods of the present disclosure. These compounds include, for example, cannabigerol; cannabichromene; cannabitriol; cannabidiol; cannabicyclolol; cannableisoin, cannabinodiol; cannabinol; delta8-tetrahydrocannabinol; delta9-tetrahydrocannabinol; cannabichromanone; cannabicoumaronone; cannabictran; 10-oxo-delta6a10a-tetrahydrocannabinol; cannabiglendol; deta7-isotetrahydrocannabinol; CBLVA; CBV; CBEVA-B; CBCVA; delta9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisoside; vitexin; canniprene; 3,4′-dihydroxy-5-methoxy bibenzyl; dihydroresveratrol; 3,4′dihydroxy-5,3′-dimethoxy-5′-isoprenyl; cannabistilbene 1; cannabistilbene 11a; cannabistilbene 11b; cannithrene 1; cannithrene 2; cannabispirone; iso-cannabispirone; cannabispirenon-A; cannabispirenone-B; cannabispiradienone; alpha-cannabispiranol; beta-cannabispiranol; acetyl-cannabispirol; 7-hydroxy-5-methoxyindan-1-spiro-cydohexane: 5-hydroxy-7-methoxyindan-1-spiro cyclohexane; myristic acid, palmitic acid, oleic acid, stearic acid, linoleic acid, linolenic acid, arachidic acid, eicosenoic acid, behenic acid, lignoceric acid, 6,7-dihydroxyindan-1-cyclohexane; cannabispiradienone; 3,4′-dihydroxy-5-methoxybibenzyl; canniprene; cannabispirone; cannithrene 1; cannithrene 2; alpha-cannabispiranol; acetyl-cannabispirol; vomifoliol; dihydrovomifoliol; beta-ionone; dihydroactinidiolide; palustrine; palustridine; plus-cannabisatvine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary metabolism in cannabis in Phytochem. Rev. DOI 10.1007/s 11101-008-9094-4).

The present disclosure provides methods for identifying compounds in hops (Humulus lupulus). These compounds include myrcene, alpha-humulene, and beta-caryophyllene, which are in hop essential oils. Other hop compounds are bitter acids, such as alpha-add and beta-add (humulone and lupulone), which are prenylated polyketide derivatives. Prenylated flavonoids are also in hops, and these include xanthohumol, desmethylxanthohumol, isoxanthohumol, 8-prenylnaringenin, and 6-prenylnaringenin (Wang et al (2008) Plant Physiol. 148:1254-1266; Nagel et al (2008) Plant Cell. 20:186-200).

The present method and system for identifying cannabinoids or terpenes in a plant extract is useful even when the extract does not include detectable cannabinoids or terpenes.

Extracting Compounds.

Extracting compounds from natural products can use methods and reagents, for example, as described by US2015/0152018 of Raber and Elzinga, which is incorporated herein by reference. Extractions can use a single step, or multiple sequential steps, and can use water, acetone, alcohol, butane, vegetable oil, mixtures thereof, and the like. Extraction methods can use chopping, shredding, homogenization, sonication, vortexing (e.g., vibrating a test tube using a vibrating rubber cup to produce a vortex), centrifugation, phase separation, filtering (e.g., paper filter, sintered glass filter, Millipore filter), incubating, heating, rotary evaporation, any combination thereof, and so on. Analytical scale methods of the present disclosure include acetone, methanol, ethanol, chloroform/methano, chloroform/ethanol, ethyl acetate, acetonitrile and so on.

Modifiers.

The present disclosure provides methods for the analysis and identification of a composition that is naturally occurring, synthetic, or a mixture of naturally occurring and synthetic compounds. Cannabinoids are a class of diverse chemical compounds that act on cannabinoid receptors in the brain. Phytocannabinoids are found in and on plants. Some commonly known phytocannabinoids include tetrahydrocannabinol (THC) and cannabidiol (CBD). Cannabinoids can also be created synthetically.

Biochemical properties of terpenes, including receptor binding, can be assessed using labeled terpenes and labeled ligands where a terpene influences binding properties of the labeled ligand. Useful labels include radioactive labels, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 6:713-728).

Gas Chromatography Terminology.

Total Flow: This is the flow into the inlet, which is the sum of the split flow and column flow.

Linear velocity: The carrier gas linear velocity or flow rate directly influences retention time and efficiency. The proper selection and setting of the carrier gas are essential to obtaining the best analysis times, efficiency and reproducibility. The carrier gas linear velocity or flow rate is controlled by adjusting the carrier gas pressure at the front of the column (commonly called the head pressure). The pressure setting is dependent on the type of carrier gas, the column length and diameter, column temperature, and the desired linear velocity or flow rate.

Purge Flow: Components of the sample that are not vaporized remain in the injector. The septum purge is a low flow which minimizes the amount of septum bleed materials which could contaminate the GC system. Septum purge gas sweeps the bottom of the septum and the top of the liner (labeled “T” for top at the GC) out through the purge vent. A typical septum purge flow is between 0.5 and 5 mL/min.

Lack of Influence of Plant Extract on Migration Time of Anchor Compounds, Plant-Derived Cannabinoids, and Plant-Derived Terpenes.

The present disclosure provides a system and method, where the presence of plant extract has no detectable influence, or has a minimal influence, on migration times of anchor compounds, or on the migration times of cannabinoids or terpenes derived from and extracted from the plant.

In preferred embodiments of the present disclosure, method, and system, there is no detectable influence on migration time, where the extract that is introduced into the GC column contains plant-derived solute that has the following weight. The solute has a total mass of over 0.01 ng (nanograms), over 0.02 ng, over 0.06 ng, over 0.10 ng, over 0.20 ng, over 0.5 ng, over 1.0 ng, over 2.0 ng, over 5 ng, over 10 ng, over 20 ng, over 50 ng, over 100 ng, over 200 ng, over 500 ng, over 1.0 ug (microgram), over 2 ug, over 5 ug, over 10 ug, over 20 ug, over 50 ug, over 100 ug, over 200 ug, over 500 ug, over 1.0 mg (milligram), over 2.0 mg, over 5.0 mg, over 10 mg, over 20 mg, over 50 mg, over 100 mg, over 500 mg, over 1,000 mg, and so on.

In a preferred embodiment, minimal influence of migration time is less than 0.2 seconds, less than 0.5 seconds, less than 1.0 seconds, less than 2.0 seconds, less than 4.0 seconds, less than 5.0 seconds, less than 6 seconds, less than 7 seconds, less than 8 seconds, less than 9 seconds, less than 10 seconds, less than 12 seconds, less than 14 seconds, less than 16 seconds, less than 18 seconds, less than 20 seconds, and so on.

Migration time differences can, without implying any limitation, be expressed as an average. For example, the present disclosure provides a method where the average difference in migration time of any given marker, with ten consecutive GC runs, is less than 0.2 seconds, less than 0.6 seconds, less than 1.0 seconds, less than 2.0 seconds, less than 4.0 seconds, less than 5.0 seconds, less than 6 seconds, less than 7 seconds, less than 8 seconds, less than 9 seconds, less than 10 seconds, less than 12 seconds, less than 14 seconds, less than 16 seconds, less than 18 seconds, less than 20 seconds, and so on. Instead of establishing this migration time using an average from ten GC runs, the present disclosure can also require that every one of ten consecutive GC runs provides a migration time that is less than one of the listed times.

Migration time differences with repeated GC runs can also be expressed in terms of difference in migration times of two markers (delta time), such as the difference between propyl benzoate and alpha pinene, or the difference between propyl benzoate and CBD, or the difference between propyl benzate and C31, or the difference between C9 and C31, and so on. As recited above, the present disclosure provides a method where the average difference in delta times, with ten consecutive GC runs, is less than 0.2 seconds, less than 0.5 seconds, less than 1.0 seconds, less than 2.0 seconds, less than 4.0 seconds, less than 5.0 seconds, less than 6 seconds, less than 7 seconds, less than 8 seconds, less than 9 seconds, less than 10 seconds, less than 12 seconds, less than 14 seconds, less than 16 seconds, less than 18 seconds, less than 20 seconds, and so on. Instead of establishing this migration time using an average, the present disclosure can also require that every one of ten consecutive GC runs provides a delta time that is less than one of the listed times.

### SUMMARY OF THE DISCLOSURE

Briefly stated, the present disclosure provides a method for using a gas chromatography (GC) apparatus and a flame ionization detector (FID), wherein the GC apparatus comprises a GC column, and wherein the GC column has a film coating that comprises phenyl groups and dimethylpolysiloxane groups, wherein the method comprises the steps of: (a) providing a plant extract that contains a plurality of analytes that comprises terpenes, cannabinoids, or both terpenes and cannabinoids, (b) combining at least two anchoring compounds with the plant extract to produce a spiked extract, (c) introducing the spiked extract into the GC apparatus, (d) initiating GC separation with a start temperature that resides in the range of 85-68 degrees C. (e) conducting a first ramp step that increases from the start temperature to a second temperature that resides in the range of 95-110 degrees C., (f) conducting a second ramp step that increases from the third temperature to a third temperature that resides in the range of 160-170 degrees C., and (g) conducting a third ramp step that increases from the fourth temperature to a fourth temperature that is at least 250 degrees C., wherein each ramp step has increases temperature at a ramp step rate (degrees/minute), and where adjacent ramp steps do not have the same ramp step rates.

In another aspect, the present disclosure provides the above method, wherein the first ramp step has a ramp step rate at about 7 degrees C. per minute, the second ramp step has a ramp step rate of about 25 degrees per minute, and the third ramp step has a ramp step rate of about 17 degrees per minute. Also provided is the above method, wherein the start temperature is at 60 degrees C., the first ramp step has a ramp step rate of 7 degrees C. per minute to 102 degrees, the second ramp step has a ramp step rate of 25 degrees per minute to 165 degrees, and the third ramp step has a ramp step rate of 17 degrees per minute to 275 degrees.

What is also embraced, is the above method, wherein the at least two anchoring compounds comprises two or more of C7, C8, C9, C10, C11, C12, C13, C14, C15, C16, C17, C18, C19, C20, C21, C22, C23, C24, C25, C26, C27, C28, C29, C30, C31 (C compounds), and propyl benzoate, and wherein the at least two anchoring compounds are separable from each other with GC, and wherein the sample analyzed by GC does not include the plant extract.

Moreover, what is encompassed is the above method, wherein the at least two anchoring compounds comprises two or more of C7, C8, C9, C10, C11, C12, C13, C14, C15, C16, C17, C18, C19, C20, C21, C22, C23, C24, C25, C26, C27, C28, C29, C30, C31 (C compounds), and propyl benzoate, and wherein the at least two anchoring compounds are separable from each other with GC, and wherein the sample analyzed by GC includes the plant extract.

In yet another aspect, what is contemplated is the above method, wherein the at least two anchoring compounds comprises two or more of C7, C8, C9, C10, C11, C12. C13, C14, C15, C16, C17, C18, C19, C20, C21, C22, C23, C24, C25, C26, C27, C28, C29, C30, and C31 (C compounds) and propyl benzoate, and wherein all of the C compounds and the propyl benzoate are separable from each other.

Also provided is the above method, wherein the at least two anchoring compounds comprises C7 and C31.

In another aspect, what is provided is the above method, wherein ten consecutive GC runs produces ten retention times for one of said anchoring compounds, wherein there is an average of the ten retention times from the ten consecutive GC runs, and wherein the difference between each of the ten retention times and the average is less than twenty seconds.

Also provided is the above method, wherein ten consecutive GC runs produces ten retention times for one of said anchoring compounds, wherein there is an average of the ten retention times from the ten consecutive GC runs, and wherein the difference between each of the ten retention times and the average is less than ten seconds.

Further embraced, is the above method, wherein ten consecutive GC runs produces ten retention times for one of said anchoring compounds, wherein the ten consecutive GC runs produces a range of retention times, wherein the range of retention times has a maximal retention time and a minimal retention time, and wherein the difference between the maximal retention time and the minimal retention time is less than twenty seconds.

Also provided, is the above method, wherein ten consecutive GC runs produces ten retention times for one of said anchoring compounds, wherein the ten consecutive GC runs produces a range of retention times, wherein the range of retention times has a maximal retention time and a minimal retention time, and wherein the difference between the maximal retention time and the minimal retention time is less than ten seconds.

In another aspect, what is provided is the above method, wherein the plant extract is from a plant that is Cannabis sativa or Humulus lupulus.

Moreover, the present disclosure provides the above method, that is capable of separating from each other, each of the compounds, alpha-pinene, myrcene, limonene, terpinolene, linalool, propyl benzoate, beta-caryophyllene, humulene, caryophyllene oxide, alpha-bisabolol, THC, CBD, C7, and C31, wherein each of said compounds has a retention time, wherein a pair of adjacently migrating compounds is defined as two compounds that have retention times that are most similar to each other, and wherein the difference in retention times between each and every one of the pairs of adjacently migrating compounds is at least 0.20 minutes.

The present disclosure provides the above method, wherein the GC column is about 30 meters long and has an internal diameter of about 0.25 millimeters.

Also contemplated is the above method, wherein the plant extract is subjected to a purification procedure to produce a purified analyte mixture, wherein the purification procedure occurs prior to adding the at least two anchoring compounds, and wherein the purified analyte mixture prior to adding the at least two anchoring compounds is sufficiently pure to introduce into the GC apparatus.

Moreover, what is provided is the above method, wherein the plant extract is combined with the at least two anchoring compounds, to produce a combination of analytes and the at least two anchoring compounds, wherein the plant extract contains an analyte mixture that is not sufficiently pure to introduce into the GC apparatus, and wherein the combination of analytes and the at least two anchoring compounds is subjected to further purification to render the combination of analytes and the at least two anchoring compounds sufficiently pure to introduce into the GC apparatus.

Also provided, is the above method, wherein the plurality of analytes comprise a mixture of terpenes and cannabinoids. In an anchoring embodiment, what is provided is the above method, wherein the at least two anchoring compounds comprises propyl benzoate.

In a GC column film embodiment, what is provided is the above method, wherein the GC column comprises a film matrix that comprises about 5% phenyl groups and about 95% dimethylpolysiloxane groups.

Also provided, is the above method wherein the plant extract is an essential oil. In a resolution embodiment, what is provided is the above method that is capable of resolving beta-caryophyllene from alpha-humulene with a difference in retention times that is greater than 0.2 minutes.

Also provided is the above method that is capable of resolving beta-caryophyllene from alpha-humulene with a difference in retention times that is greater than 0.3 minutes. In a starting temperature embodiment, what is provided is the above method, wherein the at least two anchoring compounds includes an anchoring compound that is the least retained (migrates faster) of said at least two anchoring compounds, and wherein the starting temperature is sufficiently low so that the anchoring compound that is the least retained, is less retained than all of the plurality of analytes.

In a medical assessment method embodiment, what is provided is a method for determining the most effective cannabis variety, species, or cultivar, for administering to a human subject suffering from a disorder that is one or more of neuropathic pain, cancer pain, chemotherapy-induced nausea or vomiting, spasms, and a sleep disorder, wherein the cannabis variety, species, or cultivar, has a predetermined efficacy against said disorder, wherein the method comprises the steps of: (a) Providing a sample of at least one cannabis plant, (b) Extracting said cannabis plant by an extraction procedure to provide a cannabis extract that can be used without further processing for analysis by gas chromatography (GC), wherein the analysis by gas chromatography is according to the method that is disclosed above, (c) Spiking said cannabis plant, or spiking said cannabis extract during the extraction procedure, with one or more anchor compounds, to produce a spiked cannabis extract, (d) introducing the spiked cannabis extract into the gas chromatography (GC) apparatus of the method that is disclosed above, (e) Acquiring a profile of identified cannabinoids and terpenes, wherein the profile provides cannabinoid and terpene identity and quantity, (f) Comparing the profile of identified cannabinoids and terpenes to a plurality of pre-determined cannabinoid and terpene profiles, each for a corresponding type of cannabis, (g) Determining the closest match of said profile of identified cannabinoids and terpenes with said pro-determined cannabinoid and terpene profiles, and choosing the cannabis that corresponds to said closest match of the pre-determined cannabinoid and terpene profiles, to create a chosen cannabis, and (h) Creating a record or transmission of the chosen cannabis, wherein the record or transmission exists on paper, occurs as a telephone or wireless transmission, or is stored in a computer memory.

In a system embodiment, what is provided is a system for separating and Identifying plant cannabinoids and plant terpenes derived from a plant, comprising: (a) The gas chromatography (GC) apparatus and a flame ionization detector (FID) of the above method, wherein the GC column has a film coating that comprises phenyl groups and dimethylpolysiloxane groups, and wherein the GC apparatus is capable of separating all of C7, C08, C9, C10, C11, C12, C13, C14, C15, C16, C17, C18, C19. C20, C21, C22, C23, C24, C25, C26, C27, C28, C29, C30, C31 (C compounds) and propyl benzoate from each other, wherein the separating is by the method described above, wherein the GC apparatus is programmed to perform the ramping and temperature procedures of the method described above, (b) At least one device for extracting plant cannabinoids and plant terpenes from a plant, and (c) A device for recording or transmitting information on the plant, wherein the plant has a name comprising a variety, species, or cultivar, and wherein the plant has a profile of cannabinoids and terpenes that is determinable by the method that is described above, and wherein the Information on the plant includes the name and the profile. In another system embodiment, what is provided is the above system, wherein the at least one device comprises a plant homogenizer, or wherein the at least one device comprises a centrifuge or filter for removing particulate material from a plant extract.

### BRIEF DESCRIPTIONS OF THE FIGURES

FIG. 1. Three-ramp gas chromatography method, with terpene and cannabinoid standards.

FIG. 2. Two-ramp gas chromatography method, with terpene and cannabinoid standards.

FIG. 3. Isocratic gas chromatography method, with terpene and cannabinoid standards.

FIG. 4. C based marker compounds. Separation using 3-ramp method.

FIG. 5. Cocktail 1: alpha-bisbolol, beta-caryophyllene, caryopyllene oxide, and alpha-humulene. Separation using 3-ramp method.

FIG. 6. Cocktail 2: Limonene, linalol, myrcene, alpha-pinene, beta-pinene, terpinolene, and propyl benzoate. Separation using 3-ramp method.

### ANCHORING COMPOUNDS

“Anchoring” refers to spiking a sample with two or more marker compounds of known identity. Preferably, the at least two known markers bracket most of the compounds of interest (analytes) in the sample. Operationally it is at the far ends of the chromatogram, but it is helpful to have many different known compounds. Most preferred is two markers outside of the analytes of interest, preferred is more than two spread throughout, but not necessarily evenly spaced. The anchoring compounds ensure building a dearly known and verifiably correct analytical window.

Anchoring compounds of interest include, but are not limited to, those that are disclosed herein, as well as to modified versions of these anchoring compounds, including those modified with a moiety that is methyl, ethyl, propyl, isopropyl, butyl, pentyl, hexyl and so on. The anchoring compounds used should be chosen so that it does not interfere with the detection of the analytes of interest. One or more of the anchoring compounds can be included with the plant material during extraction. Alternatively, one or more anchoring compounds can be added only after the compounds to be analyzed have been purified to the extent where they can be used for chromatographic analysis. Also, anchoring compounds can be added at both steps, that is, during extraction and also after extraction is performed.

Markets suitable for use as anchoring compounds include: C7=Heptane; C8=Octane; C9=Nonane; C10=Decane; C11=Undecane; C12=Dodecane; C13=Tridecane; C14=Tetradecane; C15=Pentadecane; C16=Cetane; C17=Heptadecane; C18=Octadecane; C19=Nonadecane; C20=Eicosane; C21=Heneicosane; C22=Docosane; C23=Tricosane; C24=Tetracosane; C25=Pentacosane; C26=Hexacosane C27=Heptacosane; C28=Octacosane; C29=Nonacosane; C30=Triacosane; C31=Hentricontane.

Number of Ramping Steps.

In a preferred embodiment, the method requires three ramping steps in gas chromatographic (GC) analysis. The present disclosure encompasses methods that require only one ramping step, only two ramping steps, only three ramping steps, only four ramping steps, only five ramping steps, and the like. In an alternative embodiment, the disclosure can exclude any method that uses one ramping step, two ramping steps, three ramping steps, four ramping steps, five ramping steps, and so on.

The present disclosure includes, without implying any limitation, a step using collected information and moving in to a software algorithm that automatically compares a database created using this chromatographic method for the purpose of chemotyping and classifying varietals of similar origin. Chromatographic methods of the present disclosure include those that are GC based, as well as by high performance liquid chromatography (HPLC), chiral HPLC, supercritical fluid chromatography (Schaffrath at al (2014) J. Chromatogr. A. 1363:270-277), and high-speed countercurrent chromatography (HSCCC) (Qiu at a (2012) J. Chromatogr. A. 124226-34).

Separability.

The present disclosure encompasses, without limitation, a method that separates at least ten compounds that are terpenes. In another embodiment, the method is capable of separating at least ten compounds that are cannabinoids. In yet another embodiment, the method is capable of separating at least ten compounds that are a mixture of terpenes and cannabinoids. Moreover, the present disclosure encompasses methods for one or more of separating, purifying, and identifying, compounds extracted from Cannabis sativa and all of the associated subspecies.

In a synthetic compound embodiment, the disclosure provides methods for one or more of separating, purifying, and identifying, compounds that are synthetic and that are created by methods of organic chemistry, including synthetic compounds that are the same as those found in Cannabis sativa and all of its associated subspecies, or from other plants, or from other natural sources. The present disclosure provides methods for identifying compounds in hops (Humulus lupulus). Cannabaceae Humulus lupulus L., and extracted compounds, have been explored for use in treating anxiety and insomnia, mild pain reduction, dyspepsia, inflammation, or liver injury (Welskirchen et al (2015) Front Physiol. 6:140. doi: 10.3389).

### Exclusionary Embodiments

In an exclusionary embodiments, the present disclosure can exclude methods for one or more of separating, purifying, and identifying, compounds extracted from a plant that is not a cannabis, that is not Cannabis sativa, that is not Cannabis indica, or that is not from a cannabis or hops. Systems and methods of the present disclosure can exclude any separation method that uses only one ramping step, only two ramping steps, only three ramping steps, only tour ramping steps, less than three ramping steps, less than two ramping steps, more than three ramping steps, and so on.

Also, what can be excluded is any method that cannot resolve all of alpha-pinene, beta-pinene, myrcene, limonene, terpinolene, linalool, propyl benzoate, beta-caryophyllene, humulene, caryophyllene oxide, alpha-bisabolol, THC, CBD, CBN, C7, and C31, when injected together as a mixture on a GC column, or when injected in as individual combinations in two or more separate injections where each injection Includes a different collection of these markers, or when injected together with a plant extract.

Also, what can be excluded is any method that cannot resolve all of the naturally-occurring alpha-pinene, beta-pinene, myrcene, limonene, terpinolene, linalool, beta-caryophyllene, humulene, caryophyllene oxide, alpha-bisabolol, THC, CBD, and CBN, that may occur in a given plant extract, and that cannot also revolve all of these compounds (the ones that detectably exist in that plant extract) from at least two markers that are spiked in the extract. The at least two markers that are spiked in the extract can be selected from propyl benzoate and from the series of long chain alkanes that is C7 to C31.

What can also be excluded is any system or method that uses GC chromatography, and where the coating, film, or matrix of the column does not comprise phenyl groups and dimethylpolysiloxane groups, or does not comprise about 5% phenyl groups and about 95% dimethylpolysiloxane groups.

Parameters that May be Used to Define Separability.

Separation, without implying any limitation, can be defined in terms of migration position of the peak signals for two adjacent compounds. For quantifying this type of separation, what is provided is a method where adjacent peak signals are separated by at least 10 seconds, by at least 20 seconds, by at least 30 seconds, by at least 40 seconds, by at least 50 seconds, by at least 1 minute, by at least 2 min, by at least 4 min, by at least 6 min, by at least 8 min, by at least 10 minutes, and the like.

Also, separation can be defined in terms of a collection of markers that is more than just two markers. Here, separation can be defined as that where each and every pair of adjacent markers has the same degree of separation that is only one of the following separations: at least 10 seconds, by at least 20 seconds, by at least 30 seconds, by at least 40 seconds, by at least 50 seconds, by at least 1 minute, by at least 2 min, by at least 4 min, by at least 6 min, by at least 8 min, by at least 10 minutes, and the like.

Alternatively, a definition of separation that applies to use of a group of more than two markers, can be that where the sum (sum is unit of time) of separation from all adjacent markers is found, and where the average is calculated (average is unit of time), and where the average is only one of the following separations: at least 10 seconds, by at least 20 seconds, by at least 30 seconds, by at least 40 seconds, by at least 50 seconds, by at least 1 minute, by at least 2 min, by at least 4 min, by at least 6 min, by at least 8 min, by at least 10 minutes, and the like.

Also, separation can be related to overlap of the trailing edge of a first compound A and the leading edge of a second compound B. For quantifying this type of separation, separation can be defined as this: region of overlap includes less than 20% of compound A and less than 20% of compound B, less than 1% of A and less than 10% of 8, less than 5% of A and less than 5% of B, less than 2% of A and less than 2% of 8, less than 1% of A and less than 1% of B, less than 0.2% of A and less than 0.2% of B, less than 0.1% of A and less than 0.1% of 8. For overlap calculations, it is assumed that a signal that is not detectably above baseline does not contain any of compound A or of compound B.

Chemistry of Matrix of Gas Chromatography (GC) Columns.

In a preferred embodiment, the method uses a highly stabilized fused silica based arylene phase via conjunctive use of methylated siloxanes to provide high resolution for hydrocarbon based compounds. The preferred chemical makeup is: 5% phenyl-arylene-95%-dimethylpolysiloxane.

In a preferred embodiment, chemical makeup inside column, which may be 5% phenyl-arylene-95%-dimethylpolysiloxane, resides in a film on the lumenal wall of the column. In other embodiments, the chemical makeup resides on a porous matrix residing within the lumen of the column. In yet other embodiments, the chemical makeup resides on beads that are packed in the column. Film thickness determines solute retention and thus solute elution temperatures. The sample capacity of the column is related to the film thickness. Thin films are faster with higher resolution, but offer lower capacity (Zebron, GC Selection Guide. Phenomenex, Inc., Torrance, Calif. (53 pages).

By way of background, gas chromatography (GC) has been used for separating organic molecules extracted from plants. Methods using only one ramping step include Tikunov et al (2005) Plant Pathol. 139:1126-1137, which used a ramp from 46 degrees to 250 degrees, Hazekamp and Fischedick (2012) Drug Test Analysis. DOI 10.1002/dta.407, which used a ramp from 60 degrees to 240 degrees, and Hillig (2004) Biochem. Systematics Ecology. 32:876-891, which used a ramp from 90 degrees to 300 degrees.

Marker Compounds.

Markers can be used for identifying unknown compounds, by establishing migration position, that is, where the unit is time or volume. Also, markers can be used for identifying quantity of compounds in the biological sample to be analyzed (analytes). Quantity can be calculated where the extinction coefficient for the marker is known and where the extinction coefficient of each analyte is known.

Standard marker compounds for the present disclosure include “C” (heptane), as well as the series of long chain alkanes that is C7 to C31. n-Hentricontane is CH₃—(CH2)₂₉—CH₃. Branched alkanes can also be used for standard markers, for example, to help the user tallor the retention time as needed. A preferred marker is propyl benzoate. In a preferred procedure, the user extracts the terpenes from the sample with a known amount of propyl benzoate in the extraction solution. In this way, the user employs a known concentration of propyl benzoate in the sample when it is injected into the machine.

Samples can also be spiked with a terpene or a cannabinoid, but only where it is known that the marker used for spiking does not overlap and does not migrate in the Immediate vicinity of the compounds to be analyzed. For example, a marker can be cannabinol (CBN). CBN is a degradation product of THC.

Starting Temperature.

A starting temperature of the present method is 60 degrees. Alternatively, a starting temperature can be a higher temperature, but the use of 80 degrees or lower starts the analysis with the more volatile components and provides a broader number of anaytes, thereby improving downstream comparatives and analytics. The use of 60 degrees as a starting temperature, enables a broader search for an anchor as it more easily includes a position for the user's anchor. The method of the present disclosure aims for verifiable accuracy and breadth of analysis.

In embodiments, the present disclosure provides methods with starting temperature of 40 degrees, 45 degrees, 50 degrees, 55 degrees, 60 degrees, 65 degrees, 70 degrees, 75 degrees, and so on. In other embodiments, what is provided is starting temperature of about 40 degrees, about 46 degrees, about 50 degrees, about 55 degrees, about 60 degrees, about 65 degrees, about 70 degrees, about 75 degrees, and so on. Also provided, is methods where the starting temperature is within the range of 50-54 degrees, 52-58 degrees, 54-56 degrees, 56-80 degrees, 58-62 degrees, 60-64 degrees, 62-66 degrees, 64-68 degrees, 68-70 degrees, 68-72 degrees, 70-74 degrees, and so on.

In exclusionary embodiments, the present disclosure can exclude any method where the starting temperature is above 60 degrees, above 62 degrees, above 64 degrees, above 66 degrees, above 68 degrees, above 70 degrees, above 72 degrees, above 74 degrees, above 76 degrees, above 78 degrees, above 80 degrees, and so on. Also, what can be excluded is any method where the starting temperature is above about 60 degrees, above about 62 degrees, above about 64 degrees, above about 66 degrees, above about 68 degrees, above about 70 degrees, above about 72 degrees, above about 74 degrees, above about 76 degrees, above about 78 degrees, above about 80 degrees, and so on. What can also be excluded, is any method where the starting temperature is below 60 degrees, below 58 degrees, below 56 degrees, below 54 degrees, below 62 degrees, below 50 degrees, below 48 degrees, below 46 degrees, below 44 degrees, below 42 degrees, below 40 degrees, and so on, as well as methods where the starting temperature is below about 60 degrees, below about 58 degrees, below about 66 degrees, below about 54 degrees, below about 52 degrees, below about 50 degrees, below about 48 degrees, below about 46 degrees, below about 44 degrees, below 42 degrees, below about 40 degrees, and so on.

Final Temperature.

The method of the present disclosure preferably has a final temperature of 250 degrees, or of about 250 degrees, or in the range of about 245-250 degrees, or in the range of about 240-250 degrees, or in the range of about 235-250 degrees, or in the range of about 230-250 degrees, or in the range of about 225-250 degrees, or in the range of about 220-250 degrees.

Also available is a final temperature of 255 degrees, or of about 250 degrees, or in the range of about 250-255 degrees, or in the range of about 245-255 degrees, or in the range of about 245-255 degrees, or in the range of about 240-255 degrees, or in the range of about 235-255 degrees.

Additionally, what is available is a final temperature of 245 degrees, or of about 245 degrees, or in the range of about 240-246 degrees, or in the range of about 235-245 degrees, or in the range of about 230-245 degrees, or in the range of about 225-245 degrees.

In exclusionary embodiments, what can be excluded is a method where final temperature is over 250 degrees, over 255 degrees, over 260 degrees, over 265 degrees, over 270 degrees, over 275 degrees, and so on. What can also be excluded is a method where final temperature is over about 260 degrees, over about 255 degrees, over about 260 degrees, over about 265 degrees, over about 270 degrees, over about 275 degrees, and so on.

An advantage of not using a final temperature of above 250 degrees, is so that the user does not heat the column as much therefore the user can cool the column faster and increase the cycle time. A goal of not using a final temperature of above 250 degrees, is that the user wants the lowest possible temperature here that allows for a clean following run (make sure everything not of interest is off of the column). An elevated temperature near the end of a run is used for ‘bake out’, which comes after analytes of interest are eluted.

Ramping Rates.

In non-limiting embodiments, the present disclosure provides one or more ramping steps in the method. Rate of ramping can be about 2 degrees per minute, about 4, about 6, about 8, about 10, about 12, about 14, about 16, about 18, about 20, about 22, about 24, about 26, about 28, about 30, about 32, about 34, about 38, about 38, about 40, about 42, about 44, about 46, about 48, about 50 degrees per minute, and so on. In other embodiments, rate of ramping can be 2-4 degrees centigrade per minute, 4-6, 6-8, 8-10, 10-12, 12-14, 14-16, 16-18, 18-20, 20-22, 22-24, 24-26, 26-30, 30-32, 32-34, 34-36, 36-38, 38-40, 40-42, 42-44, 44-48, 48-48, 48-50 degrees per minute and the like. Also, rate of ramping can be 2-6 degrees per minute, 4-8, 6-10, 8-12, 10-14, 12-16, 14-18, 16-20, 18-22, 20-24, 22-28, 24-28, 26-30, 28-32, 30-34, 32-36, 34-38, 36-40, 38-42, 40-44, 42-48, 44-48, 46-50 degrees per minute, and so on. Moreover, what is provided is rate of ramping that is 5-10 degrees per minute, 10-15, 15-20, 20-25, 25-30, 30-35, 35-40, 40-45, 45-50 degrees per minute, and so on. In exclusionary embodiments, the present disclosure provides a method that can exclude (that does not employ and that must not employ) a ramping step that uses one of the above rates.

Temperatures.

Intermediate temperature between adjacent ramping steps, where method includes a plurality of ramping steps, can be, for example, 50 degrees, 55, 60, 65, 70, 76, 80, 85, 90, 95, 100, 106, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 2856, 290, 295, 300 degrees centigrade, and so on. Also, intermediate temperature can be, for example, about 50 degrees, about 55, about 60, about 65, about 70, about 75, about 80, about 85, about 90, about 95, about 100, about 105, about 110, about 115, about 120, about 125, about 130, about 135, about 140, about 145, about 150, about 155, about 180, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300 degrees centigrade, and the like.

Correlating GC Profile with Patients Having a Specific Medical Disorder.

The present disclosure provides a system and method for correlating the GC profile for a particular sample of cannabis, with a particular patient. Cannabis occurs as many varieties, strains, species, and cultivars (see, e.g., Mandolino at a (1999) Theor. Appl. Genet. 98:86-92; Choi at al (2004) J. Nat. Prod. 67:953-9567; Novak at al (2001) Flavor Fragrance J. 16:259-262; de Meijer et al (2003) Genetics. 163:335-346). Moreover, the relative abundance of the various cannabinoids varies depending on geographic origin, soil and climate conditions, and cultivation techniques (see, e.g., Mehmedic at al (2010) J. Forensic Sci. 65:1209-1217; Hillig and Mahlberg (2004) Am. J. Bot. 91:966-975); Health Canada (February 2013) Information for Health Care Professionals. Cannabis (Marihuana, Marijuana) and the Cannabinolds (162 pages)). Terpene profile also differs, depending on the cannabis strain (see, e.g., Casano at al (2010) Acts Horticulture. 925:115-121). Recognizing that cannabis occurs as many varieties, and that each variety may have a different profile of cannabinoids and terpenes, the present disclosure provides a novel method and system for identifying which variety or cultivar of cannabis is suitable for a given patient. For example, cannabis species with higher levels of CBD were shown to have greater efficacy against Insomnia, and that Cannabis sativa asp. sativa had greater efficacy against nightmares, when compared to Cannabis sativa sap. indica (Belendiuk at al (2015) Addictive Behaviors. 60:178-181). Also, Cannabis sativa asp. indicia showed greater efficacy for Improving energy and appetite, as compared with Cannabis sativa asp. sativa (Corral (2001) J. Cannabis Therapeutics. vol. 1, issue 3-4). Cannabis, or extracts thereof, have been shown to be effective in preventing or reducing pain, sleep disturbance, and spams (see, e.g., Rog at al (2005) Neurology. 66:812-819; Wade et al (2004) Multiple Sclerosis Journal. 10:434-441).

### Examples

Terpenes that can be analyzed include alpha-bisabolol, beta-caryophyllene, alpha-humulene, limonene, linalol, myrcene, alpha-pinene, beta-pinene, and terpinolene.

Method: Terpene analysis was performed on a Shimadzu GC-2010 GC/FID with helium as the carrier gas. It was equipped with a Phenomonex ZB-5MS (30.0 m, 0.25ID, 0.25 □m) GC column. Additional columns that can be used: Agilent HP-6MS, Agilent DB-6, and Supelco SPB-5.

Three-Ramp GC Method.

The method for the 3 ramps: Start 60 degrees; Ramp at 7 degrees per minute to 102; Ramp at 25 degrees per minute to 165; Ramp at 17 degrees per minute to 275 degrees.

Two-Ramp GC Method.

The parameters for the 2 step method: Start 60 degrees C.; Ramp 25 degrees per minute to 165 degrees; Ramp to 25 degrees per minute to 275 degrees.

Isocratic GC Method.

The temperature for the isocratic method was 125 degrees C., with no change or ramp the rest of the parameters are shown below and were not changed for any of the other ramp methods either. Pressure: 66 kPa (60-70): Total Flow: 11.5 mL/min (10-13); Column Flow: 1.42 ml/min (1.3-1.5); Linear Velocity: 40.0 mL/min (25-45); Purge Flow: 3.0 mL/min (3-5); Split Ratio: 6.0 (2.5-5).

Four-Ramp GC Method.

Oven Conditions: Start Temperature 60 degrees C.

Followed by a ramp of 7 degrees per minute up to 100 degrees C.

Followed by a ramp of 25 degrees per minute up to 150 degrees C.

Followed by a ramp of 17 degrees per minute to 180 degrees C.

Followed by a ramp of 25 degrees per minute to 260 degrees C. and an additional hold time at 2500 for 7 minutes.

The oven conditions should never exceed 400 degrees C. for more than 10-20 minutes, longer than that will damage the column.

Pressure: 66 kPa (60-70)

Total Flow: 11.5 mL/min (10-13)

Column Flow: 1.42 mL/min (1.3-1.5)

Linear Velocity: 40.0 mL/min (25-45)

Purge Flow: 3.0 microliters/min (3-5)

Split Ratio: 5.0 (2.5-6)

Preparing Compounds for Analysis by Gas Chromatography (GC).

Authentic terpene standards were purchased from Sigma-Aldrich (St. Louis, Mo.). All samples and standards are prepared in ethyl acetate (EtOAc). The authentic standards were weighed out in a vial to 10-20 mg and diluted with 10 mL of EtOAc. 100 microliters was taken from the diluted sample and further diluted with 900 microliters of EtOAc to provide a final sample of 0.1-0.2 mg/mL of terpene/EtOAc. Flower samples that are tested are prepared by weighing out 350-400 mg of flower on an analytical scale and diluting with 14 mL of ethyl acetate. 2 mL is taken out, placed in a 2 mL Eppendorf tube, centrifuged for 2 min at 7000 RPM (can vary from 5000-8000) to precipitate out the particles. Finally 1 mL is transferred to a GC auto-sampler vial and placed on GC auto-sampler where its auto-injected on to the GC-FID for analysis.

Applicants devised a robust method that would separate ten terpenes, as well as the cannabinoids present, without overlapping. Applicants made two different cocktails of the terpene mixtures in order to test the ability of the method to provide baseline separation of the terpene peaks. The first mixture contained alpha-bisabolol, beta-caryophyllene, caryopyllene oxide, and alpha-humulene. The second cocktail contained limonene, linalol, myrcene, alpha-pinene, beta-pinene, terpinolene. In addition each terpene was run individually to insure the integrity of its identity (see additional information). The figures demonstrate separation of all ten terpenes, as well as an internal standard, propyl benozate. The figures also demonstrate that the novel and enhanced method of the Applicants is able to separate the cannabinoids, THC, CBD, and CBN, without overlapping with the terpenes.

Applicants were successfully able to separate all ten terpenes, as well as three cannabinoids without overlap. In addition, Applicants have three internal standards C9, C31, and propyl benzoate that will serve as anchors for the current method as well as means to quantify the terpenes present. The method can include the step of running internal standards before and within each set of runs. By running internal standards before and within each set of runs, Applicants validate that the retention times have not shifted and that the chromatography is accurate.

### DETAILED DESCRIPTION OF THE FIGURES

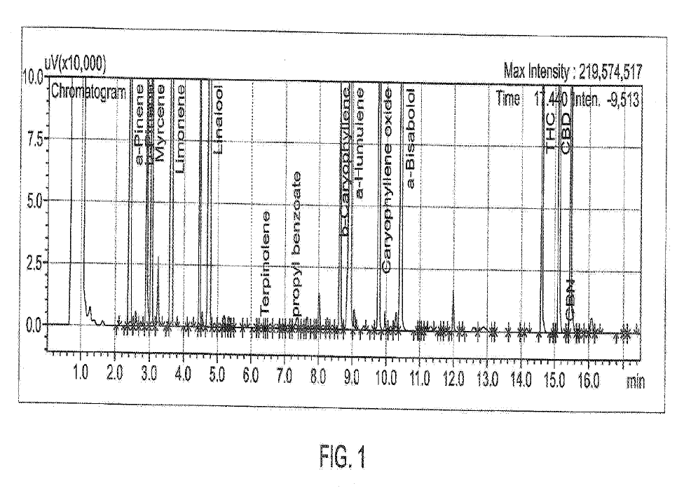

FIG. 1. Three-ramp gas chromatography method, with terpene and cannabinoid standards. Beta-caryophyllene (8.400 minutes) was well-resolved from alpha-humulene (8.770 minutes). Also, terpinolene (4.443 min) was well-resolved from linalool (4.78 min). In contrast, resolution of these compounds from each other by the 2-ramp method was poor.

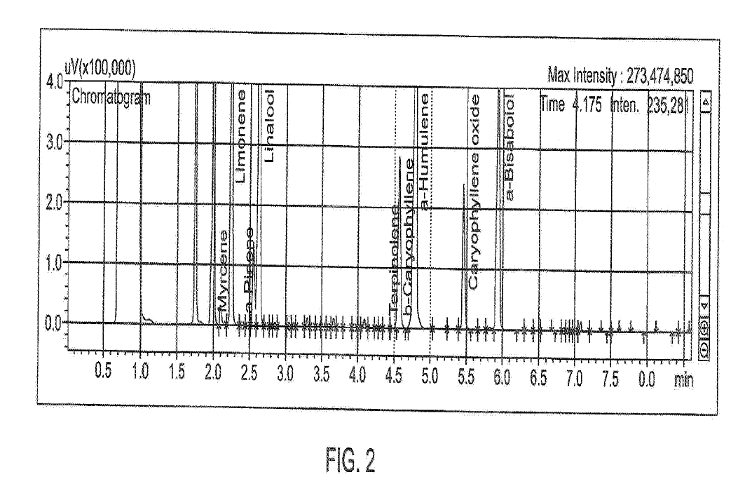

FIG. 2. Two-ramp gas chromatography method, with terpene and cannabinoid standards. Beta-caryophyllene (4.700 min) was poorly resolved from alpha-humulene (4.780 min). Also, terpinolene (2.50 min) was not well resolved from linalool (2.60 min).

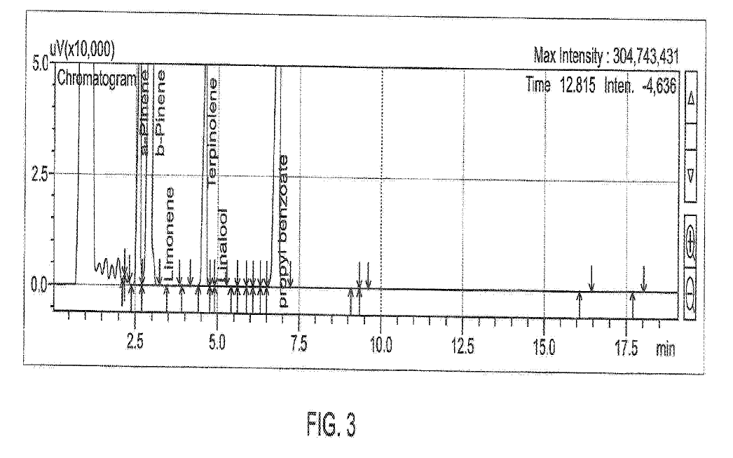

FIG. 3. Isocratic gas chromatography method, with terpene and cannabinoid standards. All of the compounds were not resolved from each other. Ten compounds, in a sample were introduced into the GC, but the result was only four peaks. Only four peaks resulted, because of poor resolution, and failure of cannabinoids to migrate through the column. Data on retention times was available for only four compounds. The cannabinoids did not come off the column, and for that reason, the isocratic method failed to give retention times for the cannabinoids.

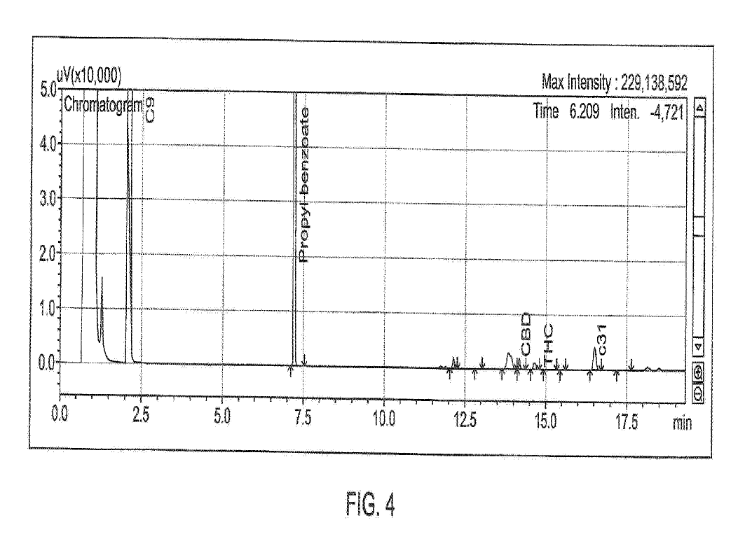

FIG. 4. Marker compounds. Separation using 3-ramp method. All three marker compounds were well-resolved from each other, and all compounds occurred as a sharp peak.

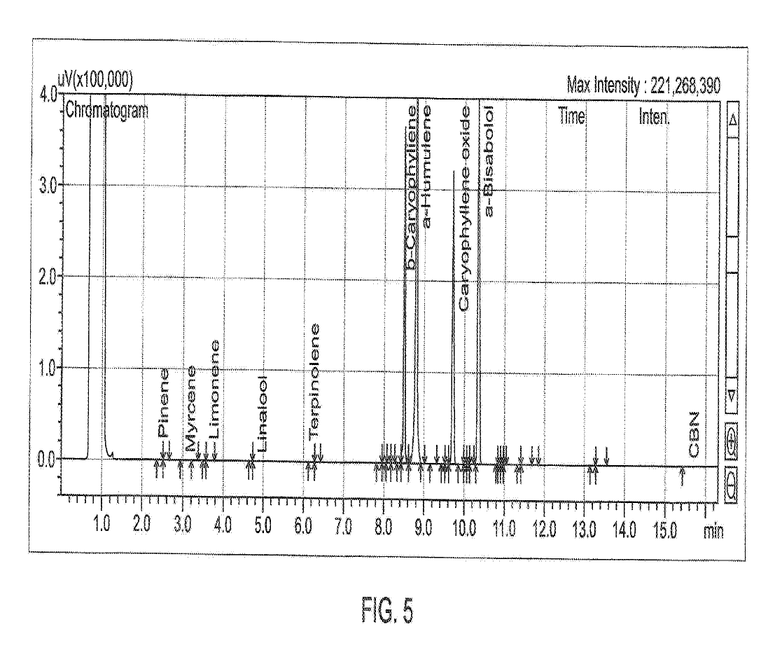

FIG. 5. Cocktail 1: alpha-bisabolol, beta-caryophyllene, caryopyllene oxide, and alpha-humulene. Separation using 3-ramp method. The GC printout shows four peaks, corresponding to beta-caryophyllene, alpha-humulene, caryophyllene oxide, and alpha-bisabolol, in this order of migration.

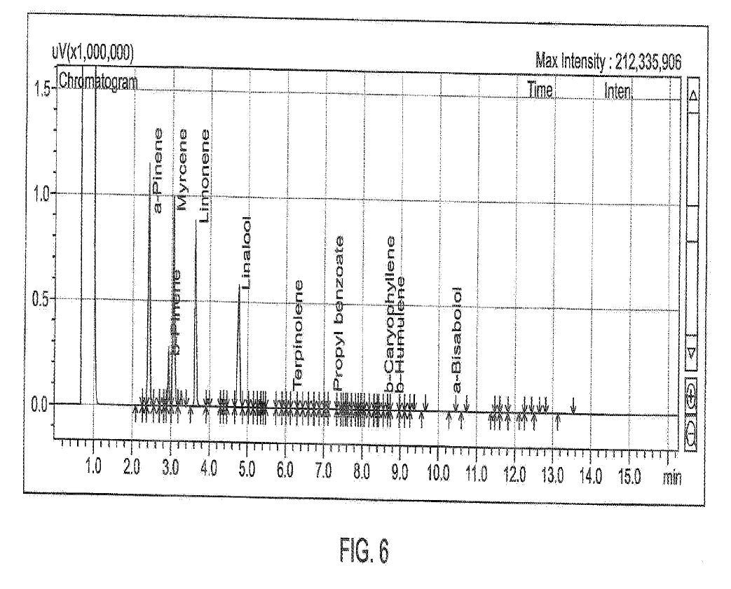

FIG. 6. Cocktail 2: Limonene, linalol, myrcene, alpha-pinene, beta-pinene, terpinolene, and propyl benzoate, Separation using 3-ramp method. The GC printout shows five peaks, alpha-pinene, beta-pinene, myrcene, limonene, and linalool, in this migration order.

Table 1. The table provides terpene profiles for several Cannabis sativa varietals. Terpene profile data from eight Cannabis sativa varietals are shown. The 3-ramp procedure was used for separation by GC chromatography.

TABLE 1
Terpene profiles using 3-ramp procedure. Profiles of terpenes from
Cannabis saliva varietals, with GC separation by 3-ramp procedure.

| | alpa- | beta-caryo- | alpha- | | |
|---|---|---|---|---|---|
| Varietal | bisabolol | phyllene | humulene | limonene | linalool |
| 1 | 1.2 | 4.80 | 1.46 | 2.76 | 1.02 |
| 2 | 0.88 | 5.79 | 2.20 | 4.73 | 0.73 |
| 3 | 0.16 | 3.56 | 1.45 | 1.32 | 0.24 |
| 4 | 0.93 | 4.96 | 1.56 | 3.75 | 0.81 |
| 5 | 0.91 | 5.70 | 2.30 | 4.41 | 0.87 |
| 6 | 0.55 | 6.15 | 2.09 | 8.99 | 2.03 |
| 7 | 0.38 | 0.83 | 0.28 | 0.91 | 1.19 |
| 8 | 0.87 | 9.05 | 2.73 | 9.64 | 2.09 |
| | | alpha- | beta- | | total |
| Varietal | myrcene | pinene | pinene | terpinoline | terpenes |
| 1 | 3.05 | 0.74 | 0.75 | 0.07 | 15.86 |
| 2 | 2.91 | 0.41 | 0.82 | 0.07 | 18.54 |
| 3 | 1.56 | 0.57 | 1.02 | 6.78 | 16.66 |
| 4 | 2.62 | 0.36 | 0.72 | 0.07 | 15.78 |
| 5 | 2.53 | 0.64 | 0.89 | 0.17 | 18.41 |
| 6 | 7.06 | 0.77 | 1.63 | 0.21 | 29.47 |
| 7 | 18.87 | 3.24 | 1.05 | 0.02 | 26.77 |
| 8 | 9.17 | 0.84 | 1.88 | 0.13 | 36.77 |

Table 2. This table reveals the retention times of standard compounds with the 3-ramp GC procedure. The 2-ramp procedure results in poor resolution of terpinolene from linalool, as compared to the 3-ramp procedure. Also, the 2-ramp procedure results in very poor resolution of beta-caryophyllene from alpha-humulene, as compared to the 3-ramp procedure.

TABLE 2
3-ramp procedure. Compounds and retention
times (minutes) using 3-ramp procedure.

| | |
|---|---|
| alpha-pinene | 2.427 |
| beta-pinene | 2.965 |
| myrcene | 3.059 |
| limonene | 3.602 |
| terpinolene | 4.443 |
| linalool | 4.779 |
| propyl benzoate | 7.800 |
| beta-caryophyllene | 8.400 |
| humulene | 8.770 |
| caryophyllene oxide | 9.700 |
| alpha-bisabolol | 10.350 |
| THC | 15.020 |
| CBD | 14.228 |
| CBN | 15.600 |
| C9 | 2.003 |
| C31 | 16.467 |

Table 3. The table discloses reveals the retention times of standard compounds with the 2-ramp GC procedure. The 2-ramp procedure results in poor resolution of terpinolene from linalool, as compared to the 3-ramp procedure. Also, the 2-ramp procedure results in very poor resolution of beta-caryophyllene from alpha-humulene, as compared to the 3-ramp procedure.

TABLE 3
2-ramp procedure. Compounds and retention
times (minutes) using 2-ramp procedure.

| | |
|---|---|
| alpha-pinene | 1.700 |
| beta-pinene | 1.900 |
| myrcene | 2.000 |
| limonene | 2.200 |
| terpinolene | 2.500 |
| linalool | 2.600 |
| propyl benzoate | 4.500 |
| beta-caryophyllene | 4.700 |
| humulene | 4.780 |
| caryophyllene oxide | 5.400 |
| alpha-bisabolol | 5.900 |
| THC | 7.200 |
| CBD | 7.200 |
| CBN | 7.200 |
| C9 | 1.545 |
| C31 | 8.458 |

Table 4. The table identifies some of the GC columns available for use with the methods of the present disclosure. ZB-35 column has a film that has 865% monomers that are —Si(methyl₂)-O— and 35% monomers that are —Si(benzyl₂)-O—. ZB-1701 has a film with 86% monomers that are —Si(methyl₂)-O— and 14% monomers that are —Si(benzyl, methyl₃-cyano)-O—.

TABLE 4
GC columns, compositions, and manufacturers (see, Zebron GC
Selection Guide. Phenomenex, Torrance, CA (53 pages).

| | Phenomenex | | |
|---|---|---|---|
| | (Zebron | | Agilent |
| Composition | columns) | J&W | Technologies |
| 100% dimethylpolysiloxane | ZB-1 | DB-1 | HP-1 |
| 100% dimethylpolysiloxane | ZB-1m8 | DB-1ms | HP-1ms |
| 5% phenyl-95% | ZB 1HT- | DB-1HT | — |
| dimethylpolysiloxane | inferno | | |
| 5% phenyl-95% | ZB-5 | DB-5 | HP-5 |
| dimethylpolysiloxane | | | |
| 5% phenyl-arylene-95% | ZB-5Msi | DB-5 | HP-5ms |
| dimethylpolysiloxane | | | |
| 5% phenyl-95% | ZB-5ms | DB-5ms | — |
| dimethylpolysiloxane | | | |
| 5% phenyl-65% | ZB-5HT- | DB-5HT | — |
| dimethylpolysiloxane | inferno | | |
| 35% phenyl-50% | ZB-35 | DB-35 | HP-35 |
| dimethylpolysiloxane | | | |
| 50% phenyl-50% | ZB-50 | DB-17 | HP-50+ |
| dimethylpolysiloxane | | | |
| 6% cyanoproypl- | ZB-624 | DB-1301 | HP-VOC |
| phenyl-94% | | | |
| dimethylpolysiloxane | | | |
| 14% cyanoproypl- | ZB-1701 | DB-1701 | — |
| phenyl-86% | | | |
| dimethylpolysiloxane | | | |
| 14% cyanopropyl- | DB-1701P | DB-1701P | — |
| phenyl-86% | | | |
| dimethylpolysiloxane | | | |
| polyethylene glycol | ZB-WAX | DB-WAXetr | HP-INNOWax |
| polyethylene glycol | ZB-WAXplus | DB-WAX | HP-20M |
| nitroterephthalic | ZB-FFAP | DB-FFAP | HP-FFAP |
| acid modified | | | |
| polyethylene glycol | | | |

It is to be understood that the present invention is not to be limited by compositions, reagents, methods, diagnostics, laboratory data, and the like, of the present disclosure, and that the present invention is not to be limited by any preferred embodiments that are disclosed herein.

What is claimed is:

1. A system for separating and identifying plant cannabinoids and plant terpenes derived from a plant, comprising:
(a) a gas chromatography (GC) apparatus and a flame ionization detector (FID), wherein the GC column has a film coating that comprises phenyl groups and dimethylpolysiloxane groups, and wherein the GC apparatus is capable of separating all of C7, C8, C9, C10, C11, C12, C13, C14, C15, C16, C17, C18, C19, C20, C21, C22, C23, C24, C25, C26, C27, C28, C29, C30, C31 (C compounds) and propyl benzoate from each other, wherein the separating is wherein the GC apparatus is programmed to perform ramping and temperature procedures,
(b) at least one device for extracting plant cannabinoids and plant terpenes from a plant, and
(c) a device for recording or transmitting information on the plant, wherein the plant has a name comprising a variety, species, or cultivar, and wherein the plant has a profile of cannabinoids and terpenes, and wherein the information on the plant includes the name and the profile.

2. The system of claim 2, wherein the at least one device comprises a plant homogenizer.

3. A method for using a gas chromatography (GC) apparatus and a flame ionization detector (FID), wherein the GC apparatus comprises a GC column, and wherein the GC column has a film coating that comprises phenyl groups and dimethylpolysiloxane groups, wherein the method comprises the steps of:
(a) providing a plant extract that contains a plurality of analytes that comprises terpenes, cannabinoids, or both terpenes and cannabinoids,
(b) combining at least two anchoring compounds with the plant extract to product a spiked extract,
(c) introducing the spiked extract into the GC apparatus,
(d) initiating GC separation with a start temperature that resides in the range of 55-65 degrees C.,
(e) conducting a first ramp step that increases from the start temperature to a second temperature that resides in the range of 95-110 degrees C.,
(f) conducting a second ramp step that increases from the third temperature to a third temperature that resides in the range of 169-170 degrees C., and
(g) conducting a third ramp step that increases from the fourth temperature to a fourth temperature that is at least 250 degrees C.,
wherein each ramp step has increases temperature at a ramp step rate (degrees/minute), and where adjacent ramp steps do not have the same ramp step rates.

4. The method of claim 1, wherein the first ramp step has a ramp step rate at about 7 degrees C. per minute, the second ramp step has a ramp step rate of about 25 degrees per minute, and the third ramp step has a ramp step rate of about 17 degrees per minute.

5. The method of claim 1, wherein the start temperature is at 60 degrees C., the first ramp step has a ramp step rate of 7 degrees C. per minute to 102 degrees, the second ramp step has a ramp step rate of 25 degrees per minute to 165 degrees, and the third ramp step has a ramp step rate of 17 degrees per minute to 275 degrees.

6. The method of claim 1, wherein the at least two anchoring compounds comprises two or more of C7, C8, C9, C10, C11, C12, C13, C14, C15, C16, C17, C18, C19, C20, C21, C22, C23, C24, C25, C26, C27, C28, C29, C30, C31 (C compounds), and propyl benzoate, and wherein the at least two anchoring compounds are separable from each other with GC, and wherein the sample analyzed by GC does not include the plant extract.

7. The method of claim 1, wherein the at least two anchoring compounds comprises two or more of C7, C8, C9, C10, C11, C12, C13, C14, C15, C16, C17, C18, C19, C20, C21, C22, C23, C24, C25, C26, C27, C28, C29, C30, C31 (C compounds), and propyl benzoate, and wherein the at least two anchoring compounds are separable from each other with GC, and wherein the sample analyzed by GC includes the plant extract.

8. The method of claim 1, wherein the at least two anchoring compounds comprises two or more of C7, C8, C9, C10, C11, C12, C13, C14, C15, C16, C17, C18, C19, C20, C21, C22, C23, C24, C25, C26, C27, C28, C29, C30, and C31 (C compounds) and propylbenzoate, and wherein all of the C compounds and the propyl benzoate are separable from each other.

9. The method of claim 1, wherein the at least two anchoring compounds comprises C9 and C31.

10. The method of claim 1, wherein ten consecutive GC runs produces ten retention times for one of said anchoring compounds,
wherein there is an average of the ten retention times from the ten consecutive GC runs, and
wherein the difference between each of the ten retention times and the average is less than twenty seconds.

11. The method of claim 1, wherein ten consecutive GC runs produces ten retention times for one of said anchoring compounds,
wherein there is an average of the ten retention times from the ten consecutive GC runs, and
wherein the difference between each of the ten retention times and the average is less than ten seconds.

12. The method of claim 1, wherein ten consecutive GC runs produces ten retention times for one of said anchoring compounds,
wherein the ten consecutive GC runs produces a range of retention times, wherein the range of retention times has a maximal retention time and a minimal retention time is less than twenty seconds.

13. The method of claim 1, wherein ten consecutive GC runs produces ten retention times for one of said anchoring compounds,
wherein the ten consecutive GC runs produces a range of retention times, wherein the range of retention times has a maximal retention time and a minimal retention time, and
wherein the difference between the maximal retention time and the minimal retention time is less than ten seconds.

14. The method of claim 1, wherein the plant extract is from a plant that is Cannabis sativa, Cannabis indica, or Humulus lupulus.

15. The method of claim 1, that is capable of separating from each other, each of the compounds, alpha-pinene, myrcene, limonene, terpinolene, linalool, propyl benzoate, beta-caryophyllene, humulene, caryophyllene oxide, alpha-bisabolol, THC, CBD, C9, and C31,
wherein each of said compounds has a retention time,
wherein a pair of adjacently migrating compounds is defined as two compounds that have retention times that are most similar to each other, and
wherein the difference in retention times between each and every one of the pairs of adjacently migrating compounds is at least 0.20 minutes.

16. The method of claim 1, wherein the GC column is about 30 meters long and has an internal diameter of about 0.25 millimeters.

17. The method of claim 1, wherein the plant extract is subjected to a purification procedure to product a purified analyte mixture,
wherein the purification procedure occurs prior to adding the at least two anchoring compounds, and
wherein the purified analyte mixture prior to adding the at least two anchoring compounds is sufficiently pure to introduce into the GC apparatus.

18. The method of claim 1, wherein the plant extract is combined with the at least two anchoring compounds, to produce a combination of analytes and the at least two anchoring compounds,
wherein the plant extract contains an analyte mixture that is not sufficiently pure to introduce into the GC apparatus, and
wherein the combination of analytes and the at least two anchoring compounds is subjected to further purification to render the combination of analytes and the at least two anchoring compounds sufficiently pure to introduce into the GC apparatus.

19. The method of claim 1, wherein the plurality of analytes comprise a mixture of terpenes and cannabinoids.

20. The method of claim 1, wherein the at least two anchoring compounds comprises propyl benzoate.

21. The method of claim 1, wherein the GC column comprises a film matrix that comprises about 5% phenyl groups and about 95% dimethylpolysiloxane groups.

22. The method of claim 1, wherein the plant extract is an essential oil.

23. The method of claim 1, wherein the at least two anchoring compounds includes an anchoring compound that is the least retained (migrates faster) of said at least two anchoring compounds, and wherein the starting temperature is sufficiently low so that the anchoring compound that is the least retained, is less retained than all of the plurality of analytes.

24. A method for classification of the efficacy of certain terpenes and cannabinoid varieties and species, or the method comprises the steps of:
(a) providing a sample of at last one cannabis plant,
(b) extracting said cannabis plant by an extraction procedure to provide a cannabis extract that can be used without further processing for analysis by chromatography, wherein the analysis by chromatography is includes,
(c) spiking said cannabis plant, or spiking said cannabis extract during the extraction procedure, with one or more anchor compounds, to produce a spiked cannabis extract,
(d) introducing the spiked cannabis extract into the chromatography system,
(e) acquiring a profile of identified cannabinoids and terpenes, wherein the profile provides cannabinoid and terpene identity and quantity,
(f) comparing the profile of identified cannabinoids and terpenes to a plurality of pre-determined cannabinoid and terpene profiles, each for a corresponding type of cannabis,
(g) determining the closest match of said profile of identified cannabinoids and terpenes with said pre-determined cannabinoid and terpene profiles, and choosing the cannabis that corresponds to said closest match of the pre-determined cannabinoid and terpene profiles, to create a chosen cannabis, and
(h) creating a record or transmission of the chosen cannabis, wherein the record or transmission exists on paper, occurs as a telephone or wireless transmission, or is stored in a computer memory.
